SEQUENCE LISTING (6 sequences)

SEQ ID NO: 1 - DNA, 20 bp, Artificial sequence V-1
  Primer to amplify a 751 bp fragment from CVH transcript.
gctcgatatgggttttggat

SEQ ID NO: 2 - DNA, 21 bp, Artificial Sequence V-2
  Primer to amplify a 751 bp fragment from CVH transcript.
ttctcttgggttccattctgc

SEQ ID NO: 3 - DNA, 20 bp, Artificial Sequence Dazl-1
  Primer to amplify a 536 bp fragment from Dazl transcript.
gcttgcatgcttttcctgct

SEQ ID NO: 4 - DNA, 19 bp, Artificial Sequence Dazl-2
  Primer to amplify a 536 bp fragment from Dazl transcript.
tgcgtcacaaagttaggca

SEQ ID NO: 5 - DNA, 21 bp, Artificial Sequence Act-RT-1
  Primer to amplify a 597 bp fragment from chicken actin transcript.
aacaccccagccatgtatgta

SEQ ID NO: 6 - DNA, 20 bp, Artificial Sequence Act-RT-2
  Primer to amplify a 597 bp fragment from chicken actin transcript.
tttcattgtgctaggtgcca

(19) United States
(12) Patent Application Publication
(10) Pub. No.: US 2014/0127812 A1
(43) Pub. Date: May 8, 2014

(54) LONG-TERM CULTURE OF AVIAN PRIMORDIAL GERM CELLS (PGCS)

(71) Applicant: SYNAGEVA BIOPHARMA CORP., LEXINGTON, MA (US)

(72) Inventors: Marie-Cecile Van de Lavoir, San Francisco, CA (US); Philip A. Leighton, San Francisco, CA (US)

(21) Appl. No.: 14/103,993

(22) Filed: Dec. 12, 2013

Related U.S. Application Data
(63) Continuation of application No. 11/049,229, filed on Feb. 1, 2005.

(51) Int. Cl. C12N 15/85

(52) U.S. Cl. CPC C12N 15/85
USPC 435/455; 435/349; 435/347

(57) ABSTRACT

The present invention is long-term cultures of avian PGCs and techniques to produce germline chimeric and transgenic birds derived from prolonged PGC cultures. In some embodiments, these PGCs can be transfected with genetic constructs to modify the DNA of the PGC, specifically to introduce a transgene encoding an exogenous protein. When combined with a host avian embryo by known procedures, those modified PGCs produce germline chimeric birds. These germline chimeric birds do not have PGC derived somatic cells or tissues. This invention includes compositions comprising long-term cultures of PGCs that can be genetically modified by gene targeting, that can accept large amounts of foreign DNA and that contribute to the germline of recipient embryos.

This invention was made with Government support under USDA SBIR 2003-33610-13933 and NIH 2 R44 HD 39583, 2 R44 GM 64261 and 2 R 44 GM 64096. The Government has certain rights in this invention.

### BACKGROUND OF THE INVENTION

Using cell culture techniques, cells of different types can be removed from animal embryos, grown in culture, and re-introduced into live embryos. When born, the resulting animal, known as a chimera, possesses characteristics of the recipient embryo and characteristics of the donor cells grown in culture. Introducing donor cells from a culture, when the donor cells have a genotype that is distinctly different from that of the recipient embryo, can be a useful technique to study the developmental biology of an organism, or to introduce selected genetic characteristics into an organism. Furthermore, because some cells can be genetically manipulated in culture, valuable animals can be created that have characteristics that reflect the genotype of the donor cells and the genotype of the recipient embryo.

The specific characteristics of an animal created from donor cells introduced into a recipient embryo depend on the type of cell maintained in culture, the specific composition and characteristics of the culture conditions, the nature of the recipient embryo, and any genetic modification introduced into the cultured cells prior to introduction into the recipient embryo. The characteristics of an animal created by introducing donor cells into a recipient embryo will also differ depending on the type of tissue in the embryo to which the donor cells contribute when introduced to the embryo during development. Germline tissue includes the sperm and eggs that carry genetic information from one generation to the next. The remaining tissue, organs, bones, etc. are known as somatic tissue and do not contribute to the germline. Accordingly, different donor cell types and different culture conditions result in different contributions to the somatic tissue or germline by the donor cells as manifested in the animal born from the recipient embryo.

Considering the characteristics and classification of the cultured donor cells, embryonic stem cells or “ES cells” can be cultured and, apparently depending on the species, can be introduced into a recipient embryo and can contribute to either the somatic or germline tissue in the resulting animal. Embryonic germ cells, or “EG cells,” can also contribute to both somatic and germline tissue. Primordial germ cells, or PGCs, on the other hand, contribute exclusively to the germline to the exclusion of somatic tissue.

Donor ES cells have been shown to contribute to the germline of offspring of chimeras created in mice but murine PGCs have not been maintained in culture and rapidly revert to EG cells that lose the restriction to the germline. In chickens, long-term cultures of ES cells contribute to somatic tissue in chimeras, and genetic modifications introduced into the genome of ES cells in culture are exhibited in somatic chimeras.

By inserting DNA constructs designed for tissue specific expression into ES cells in culture, chickens have been created that express valuable pharmaceutical products, such as monoclonal antibodies, in their egg whites. See PCT US03/25270 WO 04/015123 Zhu et al. A critical enabling technology for such animals is the creation and maintenance of truly long-term ES cell cultures that remain viable long enough for the genotype of the cloned cells to be engineered in culture.

Unlike ES cells, PGCs have only been cultured on a short-term basis and have been shown to contribute exclusively to the germline as long as the time in culture did not extend beyond a short number of days.

Long-term cultures of PGC cells have not been reported, but would be extremely useful if such cultures could be sustained for long periods of time. Typically, PGCs maintained in culture using current culture techniques do not proliferate and multiply. In the absence of robust growth, the cultures are “terminal” and cannot be maintained indefinitely. Over time, these terminal cell cultures are degraded and the cells lose their unique PGC morphology and revert to EG cells.

If the goal is to introduce a predetermined genotype into the germline of a recipient embryo, thereby enabling the animal to pass the desired genotype on to future generations, PGCs are uniquely attractive because they are known to be the progenitors of sperm and eggs. However, PGCs are notoriously difficult to grow in culture. Typically, cultures of PGCs differentiate into terminal primary cultures or they become an “immortalized” line of EG cells. Embryonic germ cells acquire a different morphology from PGCs, lose their restriction to the germline, and gain the ability to contribute to somatic tissues when injected into early stages of embryonic development. To date, long term cultures of PGCs have not been used as a vector for the introduction of foreign DNA into the genome of any organism as has been achieved with ES cells.

If long-term cultures of PGCs could be created, several important advantages would result. Cultures could be created to sustain valuable genetic characteristics of important chicken breeding lines that are relied upon in the poultry and egg production industries. Currently, extraordinary measures are undertaken to prevent valuable breeding lines from being lost through accident or disease. These measures require maintaining large numbers of members of a line as breeding stock and duplicating these stocks at multiple locations throughout the world. Maintaining large numbers of valuable animals in reserve is necessary, because preserving genetic diversity within a breeding line is also important. If the valuable genetic characteristics of these birds could be preserved in PGC cell cultures, especially if the PGC cultures could be stored in liquid nitrogen, the expense of large scale reserve breeding populations could be avoided.

Long term cultures of PGCs would also be highly valuable for the production of pharmaceutical products from the eggs of genetically engineered chickens. Producing genetically engineered chickens using PGCs requires introducing genetic modifications into the genotype of the PGCs while they are maintained in culture. Techniques for a wide variety of genetic manipulations of target cells in culture are well known. However, one main difficulty is that to alter the genotype of PGCs in culture, the culture must remain viable for a length of time that is longer than the existing culture techniques allow.

Performing genetic engineering of cells in culture requires that the ideal culture conditions be maintained while genetic modifications are introduced, while cells containing the genetic modifications are selected, and while the selected cells grow and proliferate in culture. Cells that are capable of proliferating are distinguished by their ability to generate large numbers of cells (e.g. 10⁴ to 10⁷ cells) within several days to several weeks following clonal or nearly clonal derivation. The founder cells will be the rare cells that carry the genetic modification that is desired. Typically, these cells are generated in culture at frequencies of 10⁻⁴ to 10⁻⁷ following the application of technologies for genetic modification that are well known, (e.g. lipofection or electroporation). Therefore, production of cells in culture requires passaging the cells to provide space and nutrients for the cells to proliferate and generate a sufficient number of cells to allow selection of the rare, genetically-modified cells in culture.

In addition, the culture conditions must be sufficiently robust to allow the cells to grow from an individual genetically-modified cell into a colony of 10⁴ to 10⁷ cells to be used for genetic analysis in vitro and for the production of chimeras. Thus, if the length of the culture could be extended while preserving the cells as true PGCs, the cells could be engineered and introduced into recipient embryos at a point in embryonic development when the germline competent cells are migrating to the gonad. These engineered PGCs would contribute exclusively to the nascent population of spermatogonia or oogonia (i.e., the sperm and eggs) in the resulting animals upon maturity. In such a resulting animal, the entirety of the somatic tissue would be derived from the recipient embryo and the germline would contain contributions from both the donor cells and the recipient embryos. Because of the mixed contribution to the germline, these animals are known as “germline chimeras.” Depending on the extent of chimerism, the offspring of germline chimeras will be derived either from the donor cell or from the recipient embryo.

In some cases, the cultured cells may contribute to the germline of both male and female recipients. For example, chicken blastodermal cells from males will contribute to the female germline and vice-versa (Kagami et al., (1996) Molecular Reproduction and Development 42, 379-387). When primordial germ cells are transferred from one embryo to another, contributions to the germline are frequent when male cells are inserted into male recipients and when female cells are inserted into female recipients. However, male PGCs colonized the germline of female recipients only very infrequently and the colonization of female PGCs in a male recipient was also a rare event (Naito et al., (1999). Journal of Reproduction and Fertility 117, 291-298).

Several attempts to establish lines of chicken PGCs have been reported but none of these attempts has yielded a line of cells that could be sustained. In each of these cases, the culture of PGCs has differentiated into EG cells (WO 00/47717; WO99/06533; WO99/06534; Park et al., (2003) Mol Reprod Dev 65, 389-395; Park and Han, (2000) Mol Reprod Dev 56, 475-482) or cells with an ES cell phenotype (WO 01/11019). In other cases, PGC cultures could be maintained for only 5 days (Chang et al., (1997) Cell Biology International 21, 495-499; Chang et al., (1995) Cell Biology International 19, 569-576) or 10 days (Park et al., (2003) Biol Reprod 68, 1657-1662). In another case, PGCs were maintained in culture for 2 months, but the cells proliferated only very slowly and the culture could not be passaged (Han et al., (2002) Theriogenology 58, 1531-1539).

The ability of PGC cell cultures to proliferate is essential for selection of cells whose genome has been altered by random integration of a transgene or by site-specific modification. In both types of genetic modification, the proportion of cells acquiring the genetic modification as a stable integration into the genome of the cell in culture is very low on the order of one cell in between ten thousand and one cell in a million (i.e. 1×10⁻⁴ to 1×10⁻⁶). Accordingly, the ability to establish a rapidly growing culture is required to obtain an adequate population of cells derived from the rare event that creates the genetic modification in the genome of a cell in culture.

Chicken primordial germ cells have been genetically modified using a retroviral vector within a few hours following isolation from Stage 11-15 embryos (Vick et al., (1993) Proc. R. Soc. Lond. B 251, 179-182). However, the size of the transgene is limited to less then 8 kb and site-specific changes to the genome cannot be executed using this technology. Stable changes to the genome of cultured PGCs have not been reported previously.

The avian egg offers an ideal repository for biologically active proteins and provides a convenient milieu from which proteins can be isolated. Avian animals are also attractive candidates for a broad variety of transgenic technologies. However, application of the full range of mammalian transgenic techniques to avian species has been unsuccessful due to the absence of a cultured cell population into which genetic modifications can be introduced and transmitted into the germline. To date, genetically transfected PGCs have not been maintained in culture nor used to transmit genetic modifications performed in culture.

### SUMMARY OF INVENTION

This invention relates to long-term cultures of avian primordial germ cells (PGCs) and several additional inventions enabled by the creation of a long-term culture where avian PGCs proliferate and where PGC cultures can be extended through multiple passages to extend the viability of the culture beyond 40 days, 60 days, 80 days, 100 days, or longer. The PGCs of the invention proliferate in long term cultures and produce germline chimeras when injected into recipient embryos.

The PGCs maintained in the culture described herein maintain a characteristic PGC morphology while maintained in culture. The PGC morphology may be observed by direct observation, and the growth of cells in culture is assessed by common techniques to insure that the cells proliferate in culture. Cell cultures that proliferate are defined as non-terminal and are observed to have a greater number of cells in culture at the latter of 2 distinct time points. The PGCs in the culture of the invention may have 1×10⁵ or more cells in any particular culture and this number may be observed to increase over time. Accordingly, one aspect of the invention is the observation of a proliferating PGC culture that contains a larger number of cells after a period of days, weeks, or months compared to an earlier time point in the life of the culture. Ideally, the culture contains at least 1×10⁵ cells and may be observed to have a higher number after any length of time growing in culture. Furthermore, the PGCs may be observed to be the dominant species in the culture such that, when considering the minimal contribution made by non-chicken feeder cells, the proliferating component of the cell culture consists essentially of chicken primordial germ cells, to the substantial exclusion of other chicken-derived cells.

The culture also manifests the characteristic of allowing proliferation by passage such that samples or aliquots of cells from an existing culture can be separated and will exhibit robust growth when placed in new culture media. By definition, the ability to passage a cell culture indicates that the cell culture is growing and proliferating and is non-terminal. Furthermore, the cells of the invention demonstrate the ability to create germline chimeras after several passages and maintain a PGC morphology. As described herein, this proliferation is an essential feature of any cell culture suitable for stable integration of exogenous DNA sequences.

The PGCs of the invention can be obtained by any known technique and grows in the culture conditions described herein. However, it is preferred that whole blood is removed from a stage 15 embryo and is placed directly in the culture media described below. This approach differs from other approaches described in the literature wherein PGCs are subjected to processing and separation steps prior to being placed in culture. Unlike conventional culture techniques, the culture and methodology of the present invention relies on robust differential growth between PGCs and other cells from whole blood that may initially coexist in the medium, in order to provide the large populations of PGCs in culture described here. Accordingly, the present invention provides culture of PGCs derived directly from whole blood that grow into large cell concentrations in culture, can go through an unlimited number of passages, and exhibit robust growth and proliferation such that the PGCs in culture are essentially the only cells growing and proliferating. These culture conditions provide an important advantage of the present invention, thereby rendering the collection, storage, and maintenance of PGCs in culture particularly simple and efficient and providing a readily available source of donor cells to create germline chimeras that pass the genotype of cultured PGC cells to offspring.

The PGCs maintained in culture by the inventors have demonstrated the existence of a non-terminal culture and have currently existed for at least 327 days in culture. These cells are growing and proliferating in the same manner as was observed at 40, 60, 80, or 100 days (and all integral values therein) and the cells continue to contribute to germline chimeras as described below, and thus, exhibit the primary distinguishing characteristics of true PGCs, i.e., the exclusive contribution to the germline when introduced into a recipient embryo. The culture methodology of the invention includes using whole blood, which contains red blood cells and other metabolically active cell types, placing a mixture of cells into culture along with primordial germ cells and allowing the culture to evolve to consist essentially of avian PGCs. The cell culturing technology of the invention avoids any cell separation processes or techniques and relies on differential growth conditions to yield the predominance of PGCs in culture.

The culture medium is conditioned with BRL (Buffalo Rat Liver cells), contains fibroblast growth factors, stem cell factor, and chicken serum. The particular characteristics of the medium are described in greater detail below.

In one aspect of the present invention, a culture is established that has a large number of PGCs that are genetically identical and which proliferate to yield a long-term cell culture. These PGCs can be used repeatedly to create germline chimeras by introducing the PGCs from culture to recipient embryos. In previous attempts to use PGCs to create germline chimeras, the number of chimeras that could be created was inherently limited by the inability to grow long-term cultures of true PGCs that retain the PGC phenotype. Because long-term cultures are enabled by the present invention, any number of germline chimeras can be created from the same cell culture and an entire population of germline chimeras can be established having genetically identical, PGC-derived germlines. Accordingly, one aspect of the present invention is the creation of large numbers, including greater than 3, greater than 4, greater than 5, 10, 15 and 20 germline chimeric animals all having genetically identical PGC-derived cells in their germline. Another aspect of the invention is the creation of a population of germline chimeras having genetically identical PGC-derived cells in their germline that have, within the population, age differentials that reflect the use of the same long-term cell culture to create germline chimeras. The age differentials exceed the currently available ability to culture primordial germ cells over time and are 40 days or more. Accordingly, the present invention includes two or more germline chimeras having identical PGC-derived cells in their germline that differ in age by more than 40 days, 60 days, 80 days, 100 days, etc., or any other integral value therein. The invention also includes the existence of sexually mature germline chimeras having genetically identical PGC-derived cells in germline, together with the existence of a non-terminal PGC culture used to create these germline chimeras and from which additional germline chimeras can be created.

Because the PGCs can be maintained in culture in a manner that is extremely stable, the cells can also be cryo-preserved and thawed to create a long-term storage methodology for creating germline chimeras having a capability to produce offspring defined by the phenotype of the PGCs maintained in culture.

The capability to produce large numbers of germline chimeras also provides the ability to pass the PGC-derived genotype through to offspring of the germline chimera. Accordingly, the present invention includes both populations of germline chimeras having genetically identical PGC-derived cells in the germline, but also offspring of the germline chimeras whose genotype and phenotype is entirely determined by the genotype of the PGCs grown in culture. Thus, the invention includes the offspring of a germline chimera created by germline transmission of a genotype of a primordial germ cell held in culture. Accordingly, the invention includes each of the existence of a primordial germ cell culture containing PGCs of a defined phenotype, a germline chimera having the same primordial germ cells as part of its germline, and an offspring of the germline chimera having a genotype and phenotype dictated by the PGCs in culture.

As has been described previously, the existence of long-term PGC cultures enables the ability to stably transfect the cells in culture with DNA encoding exogenous proteins or introducing other desirable genetic manipulations such as gene insertions and knock-outs of a transgenic animal. Accordingly, all of the above-described populations of PGCs in culture, germline chimeras, and offspring of germline chimeras can also be comprised of a DNA construct stably integrated into the genome of the primordial germ cell, transmitted into the germline of the germline chimera, and transmitted into future generations comprised of offspring of the germline chimeras.

The primordial germ cells may contain virtually any engineered genetic constructs and may be used to introduce genetic modifications into birds that exceed the size restrictions currently imposed by retroviral technologies, including the site-specific insertion of transgenes encoding full length exogenous proteins such as monoclonal antibodies. In a preferred embodiment, genetically engineered chickens express exogenous proteins in a tissue specific fashion in the oviduct to express exogenous proteins in the egg.

The PGC cultures of the invention are sufficiently stable to allow a transgene to become stably integrated into the genome of the PGC, to isolate the genetically modified cells from non-modified cells in the culture, and to introduce the modified cells into a recipient embryo, while maintaining the ability of the cultured PGCs to contribute to the germline in a resulting chimera. In cases where expression of the transgene is controlled by a tissue specific promoter, the transgene would not be expressed in PGCs. In these cases, the transgene would be expressed in the selected tissues in transgenic offspring of the germline chimera. Whole genomes can be transferred by cell hybridization, intact chromosomes by microcells, subchromosomal segments by chromosome mediated gene transfer and DNA fragments in the kilobase range by DNA mediated gene transfer (Klobutcher, L. A. and F. H. Ruddle, Ann. Rev. Biochem., 50: 533-554, 1981). Intact chromosomes may be transferred by microcell-mediated chromosome transfer (MMCT) (Fournier, R. E. and Ruddle, F. H., Proc. Natl. Acad. Sci., USA 74: 319-323, 1977).

Stable long-term cultures of PGCs that yield genetically engineered chickens are necessary for several applications in avian transgenesis, including the production of proteins for the pharmaceutical industry, production of chickens that deposit human monoclonal antibodies in their eggs, and to make site-specific changes to the avian genome for any number of other applications including human sequence polyclonal antibodies.

The ratio of donor-derived and recipient-derived PGCs in a recipient embryo can be altered to favor colonization of the germline in PGC-derived chimeras. In developing chicken and quail embryos, exposure to busulfan either greatly reduces or eliminates the population of primordial germ cells as they migrate from the germinal crescent to the gonadal ridge (Reynaud (1977a) Bull Soc Zool Francaise 102, 417-429; Reynaud (1981) Arch Anat Micro Morph Exp 70, 251-258; Aige-Gil and Simkiss (1991) Res Vet Sci 50, 139-144). When busulfan is injected into the yolk after 24 to 30 hours of incubation and primordial germ cells are re-introduced into the vasculature after 50 to 55 hours of incubation, the germline is repopulated with donor-derived primordial germ cells and subsequently, donor derived gametes are produced (Vick et al. (1993) J Reprod Fert 98, 637-641; Bresler et al. (1994) Brit Poultry Sci 35 241-247).

Methods of the invention include: obtaining PGCs from a chicken, such as from the whole blood of a stage 15 embryo, placing the PGCs in culture, proliferating the PGCs to increase their number and enabling a number of passages, creating germline chimeras from these long-term cell cultures, and obtaining offspring of the germline chimeras having a genotype provided by the cultured PGCs. The methods of the invention also include inserting genetic modifications into a population of PGCs in culture to create stably transfected PGCs, selecting cells from this population that carry stably integrated transgenes, injecting the genetically modified cells carrying the stably integrated transgenes into a recipient embryo, developing the embryo into a germline chimera containing the genetic modification in the germline, raising the germline chimera to sexual maturity and breeding the germline chimera to obtain genetically modified offspring wherein the genetic modification is derived from the cultured PGC.

### DESCRIPTION OF THE FIGURES

FIG. 1A: PGCs maintained in culture for 54 days. Note that the cells are not attached and maintain a round morphology. Arrows indicate several dividing cells visible in this culture.

FIG. 1B: PGCs maintained in culture for 234 days. These cells are cultured on a feeder layer of irradiated STO cells.

FIG. 2: Gene expression as determined by RT-PCR of the germ cell markers CVH and Dazl. Cells were in culture for 32 days. Lane 1 shows expression of both CVH and Dazl in an aliquot of PGCs. A second sample, in lane 2, did not have sufficient mRNA as determined by the absence of actin. CES cells were also analyzed; actin was expressed but the cES cells did not express CVH and Dazl was expressed only weakly.

FIG. 3: Western analysis of PGCs maintained in culture for 166 days. Testis was used as positive control and liver as a negative control. Rabbit anti-chicken CVH IgG was used as the primary antibody.

FIG. 4: Telomeric Repeat Amplification Protocol (TRAP) Assay. Different dilutions of cell extracts of 2 different PGC cell lines (13&16) maintained in culture for 146 days. The positive control consisted of the transformed human kidney cell line 293 and the negative control was lysis buffer only with no template added. In the PGC and positive control lanes, repeat sequences are visible indicating the presence of telomerase.

FIG. 5A: cEG cells derived from PGCs maintained in culture. 5B: Chicken embryonic stem cells. Note the small cells, big nucleus (light grey) and pronounced nucleolus in both cell types.

FIG. 6: Chimeras obtained from cEG cells derived from PGCs. The EG cells were derived from black feathered Barred Rock embryos. As recipients, a white feathered (White Leghorn) embryo was used. Somatic chimerism is evident by the black feathers.

FIG. 7. Rooster IV7-5 with his offspring. A White Leghorn is homozygous dominant at the dominant, white locus (I/I). When bred to a Barred Rock hen (i/i) all offspring from a White Leghorn will be white (I/i). A black chick demonstrates that the injected PGCs (derived from a Barred Rock embryo (i/i)) have entered the germline of the White Leghorn rooster.

FIG. 8. Southern analysis of cx-neo transgene in a line of primordial germ cells (PGCs).

### DETAILED DESCRIPTION OF INVENTION

As used herein, the terms chicken embryonic stem (cES) cells mean cells exhibiting an ES cell morphology and which contribute to somatic tissue in a recipient embryo derived from the area pellucida of Stage X (E-G&K) embryos (the approximate equivalent of the mouse blastocyst). CES cells share several in vitro characteristics of mouse ES cells such as being SSEA-1⁺, EMA-1⁺ and telomerase⁺. ES cells have the capacity to colonize all of the somatic tissues.

As used herein, the terms primordial germ cells (PGCs) mean cells exhibiting a PGC morphology and which contribute exclusively to the germline in recipient embryos, PGCs may be derived from whole blood taken from Stage 12-17 (H&H) embryos. A PGC phenotype may be established by (1) the germline specific genes CVH and Dazl are strongly transcribed in this cell line, (2) the cells strongly express the CVH protein, (3) the cells do not contribute to somatic tissues when injected into a Stage X nor a Stage 12-17 (H&H) recipient embryo, (4) the cells give rise to EG cells (see below), or (5) the cells transmit the PGC genotype through the germline when injected into Stage 12-17 (H&H) embryos (Tajima et al. (1993) Theriogenology 40, 509-519; Naito et al., (1994) Mol Reprod Dev, 39, 153-161; Naito et al., (1999) J Reprod Fert 117, 291-298).

As used herein, the term chicken embryonic germ (cEG) cells means cells derived from PGCs and are analogous in function to murine EG cells. The morphology of cEG cells is similar to that of cES cells and cEG cells contribute to somatic tissues when injected into a Stage X (E-G&K) recipient.

The germline in chickens is initiated as cells from the epiblast of a Stage X (E-G & K) embryo ingress into the nascent hypoblast (Kagami et al., (1997) Mol Reprod Dev 48, 501-510; Petitte, (2002) J Poultry Sci 39, 205-228). As the hypoblast progresses anteriorly, the pre-primordial germ cells are swept forward into the germinal crescent where they can be identified as large glycogen laden cells. The earliest identification of cells in the germline by these morphological criteria is approximately 8 hours after the beginning of incubation (Stage 4 using the staging system established by Hamburger and Hamilton, (1951) J Morph 88, 49-92). The primordial germ cells reside in the germinal crescent from Stage 4 (H&H) until they migrate through the vasculature during Stage 12-17 (H&H). At this time, the primordial germ cells are a small population of about 200 cells. From the vasculature, the primordial germ cells migrate into the genital ridge and are incorporated into the ovary or testes as the gonad differentiates (Swift, (1914) Am J Anat 15, 483-516; Meyer, (1964) Dev Biol 10, 154-190; Fujimoto et al. (1976) Anat Rec 185, 139-154).

The following describes the unexpected finding that PGCs can be isolated from the blood of Stage 12-17 (H&H) embryos, that the cells will proliferate rapidly and maintain their PGC phenotype, that the PGCs can be passaged at daily or 2-day intervals, that the PGCs will colonize the germline but not somatic tissues after at least 110 days in culture, that viable offspring can be obtained from cells that have been in culture for 110 days, that the PGCs can be genetically modified by transfection with a transgene, and that the genetically modified PGCs can be isolated and propagated into a colony of genetically modified PGCs.

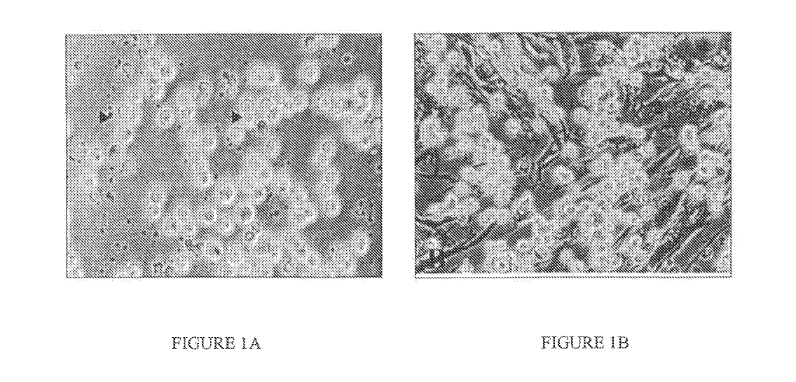

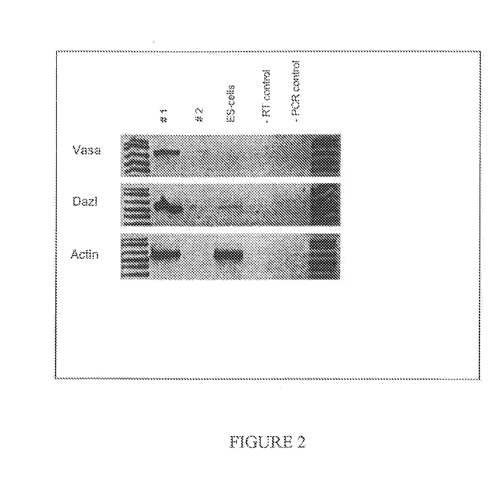

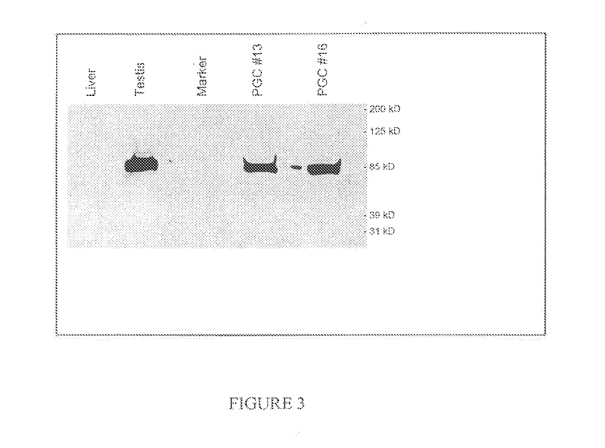

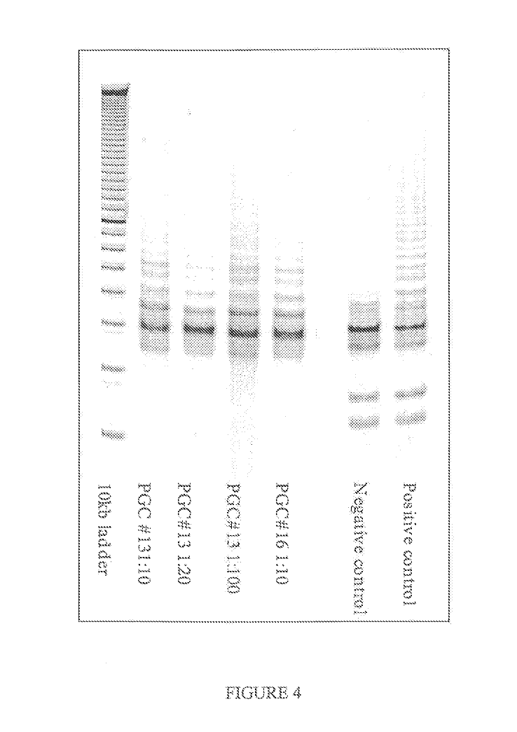

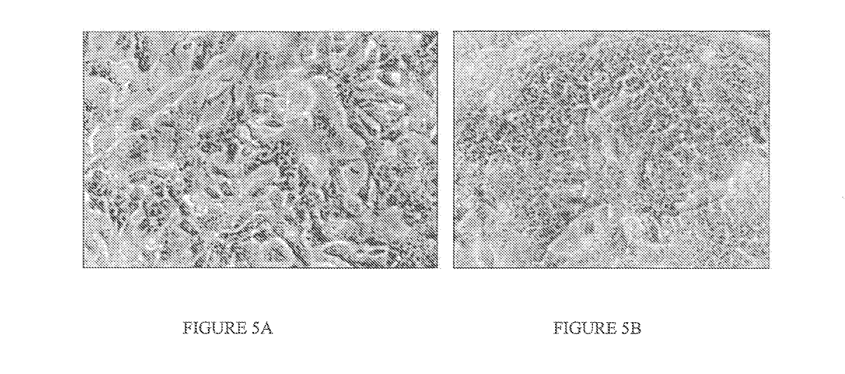

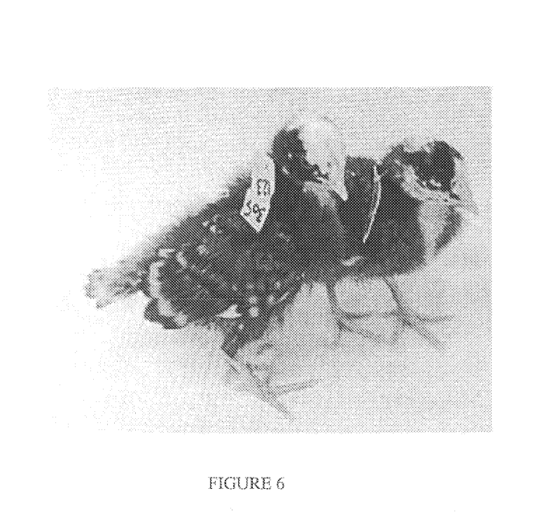

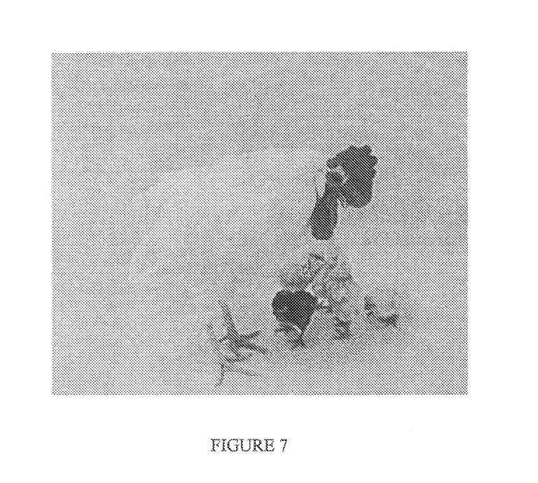

Pursuant to this invention, chicken PGC cell lines have been derived from blood taken from Stage 16 (H&H) embryos that have a large, round morphology (FIG. 1). These cells are confirmed to be chicken PGCs by their morphology after long term culturing and their ability to yield PGC-derived offspring. In addition, the PGC cultures express the germline-specific genes Dazl and Cvh (FIG. 2) and the Cvh protein is produced by the cells in culture (FIG. 3). PGCs in culture also express telomerase (FIG. 4) indicating that they have an immortal phenotype. Furthermore, PGCs will give rise to embryonic germ (EG) cells in the appropriate culture conditions (FIG. 5). By analogy, mouse and human PGCs will give rise to EG cells when treated in an analogous fashion. Mouse EG cells will contribute to somatic tissues and chicken EG cells also contribute to somatic tissues as indicated by black feather pigmentation in chimeras (FIG. 6). Chicken PGCs have been genetically modified as indicated by Southern analysis (FIG. 7). In a preferred embodiment, the CX promoter is stably integrated into the genome of a PGC and is used to facilitate expression of the gene encoding aminoglycoside phosphotransferase (APH) which is also integrated into the genome of a PGC and is used to confer resistance to neomycin added to culture media in order to select PGCs that have been genetically modified.

### Example 1

### Derivation of Cultures of Chicken PGCs

Two to five μL of blood taken from the sinus terminalis of Stage 15-17 (H&H) embryos were incubated in 96 well plates in a medium containing Stem Cell Factor (SCF; 6 ng/ml or 60 ng/ml), human recombinant Fibroblast Growth Factor (hrFGF; 4 ng/ml or 40 ng/ml), 10% fetal bovine serum, and 80% KO-DMEM conditioned medium. The wells of the 96-well plates was seeded with irradiated STO cells at a concentration of 3×10⁴ cells/cm².

KO-DMEM conditioned media were prepared by growing BRL cells to confluency in DMEM supplemented with 10% fetal bovine serum, 1% pen/strep; 2 mM glutamine, 1 mM pyruvate, 1X nucleosides, 1X non-essential amino acids and 0.1 mM β-mercaptoethanol and containing 5% fetal bovine serum for three days. After 24 h, the medium was removed and a new batch of medium was conditioned for three days. This was repeated a third time and the three batches were combined to make the PGC culture medium.

After approximately 180 days in culture, one line of PGCs was grown in media comprised of 40% KO-DMEM conditioned media, 7.5% fetal bovine serum and 2.5% chicken serum. Under these conditions, the doubling time of the PGCs was approximately 24-36 hours.

When the culture was initiated, the predominant cell type was fetal red blood cells. Within three weeks, the predominant cell type was that of a PGC. Two PGC cell lines were derived from 18 cultures that were initiated from individual embryos.

A line of PGCs has been in culture for over 9 months, maintain a round morphology, and remain unattached (FIGS. 1A &B). PGCs have been successfully thawed after cryopreservation in CO₂ independent medium containing 10% serum and 10% DMSO.

### Example 2

### Cultured PGCs Express Cvh and Dazl

Expression of CVH, which is the chicken homologue of the germline specific gene VASA in Drosophila, is restricted to cells within the germline of chickens and is expressed by approximately 200 cells in the germinal crescent (Tsunekawa et al., 2000). CVH expression is required for proper function of the germline in males; loss of CVH function causes infertility in male mice (Tanaka et al., 2000). The expression of Dazl is restricted to the germline in frogs (Houston and King, 2000) axolotl (Johnson et al., 2001), mice (Schrans-Stassen et al., 2001), rat (Hamra et al., 2002), and human (Lifschitz-Mercer et al., 2000). Deletion of Dazl led to spermatogenic defects in transgenic mice (Reijo et al., 1995).

After 32 days, PGCs were washed with PBS, pelleted and mRNA was isolated from the tissue samples with the Oligotex Direct mRNA kit (Qiagen). cDNA was then synthesized from 9 μl of mRNA using the SuperScript RT-PCR System for First-Strand cDNA synthesis (Invitrogen). Two μl of cDNA was used in the subsequent PCR reaction. Primer sequences which were derived from the CVH sequence (accession number AB004836), Dazl sequence (accession number AY211387), or β-actin sequence (accession number NM_{—}205518) were:

V-1
(SEQ ID NO. 1)
GCTCGATATGGGTTTTGGAT
V-2
(SEQ ID NO. 2)
TTCTCTTGGGTTCCATTCTGC
Dazl-1
(SEQ ID NO. 3)
GCTTGCATGCTTTTCCTGCT
Dazl-2
(SEQ ID NO. 4)
TGC GTC ACA AAG TTA GGC A
Act-RT-1
(SEQ ID NO. 5)
AAC ACC CCA GCC ATG TAT GTA
Act-RT-2
(SEQ ID NO. 6)
TTT CAT TGT GCT AGG TGC CA

Primers V-1 and V-2 were used to amplify a 751 bp fragment from the CVH transcript. Primers Dazl-1 and Dazl-2 were used to amplify a 536 bp fragment from the Dazl transcript. Primers Act-RT-1 and Act-RT-R were used to amplify a 597 bp fragment from the endogenous chicken β-actin transcript. PCR reactions were performed with AmpliTaq Gold (Applied Biosystems) following the manufacturer's instructions.

### Example 3

### PGCs Express the Cvh Protein

Protein was extracted from freshly isolated PGCs using the T-Per tissue protein extraction kit (Pierce). Protein from cells was extracted by lysing the cells in 1% NP₄O; 0.4% deoxycholated 66 mM EDTA; 10 mM, Tris, pH7.4. Samples were run on 4-15% Tris-HCL ready gel (Bio-Rad). After transfer onto a membrane, Western blots were performed with Super Signal West Pico Chemiluminescent Substrate kits (Pierce) as instructed. A rabbit anti-CVH antibody was used as a primary antibody (1:300 dilution) and a HRP-conjugated goat anti-rabbit IgG antibody (Pierce, 1:100,000) was used as a secondary antibody (FIG. 3).

### Example 4

### Cultured PGCs Express Telomerase

Primordial germ cells were trypsinized, pelleted and washed with PBS before being frozen at −80° C. until analysis. Cell extracts were prepared and analyzed according to the manufacturer's directions using the TRAPeze Telomerase Detection Kit (Serologicals Corporation) which is based upon the Telomeric Repeat Amplification Protocol (TRAP) (Kim et al., 1994). FIG. 4.

### Example 5

### Embryonic Germ (EG) Cells can be Derived from Cultures of PGCs

Chicken EG cells have been derived from PGCs by allowing the cells to attach to the plate and subsequently removing FGF, SCF and chicken serum; these conditions are the same as those for ES cell culture. The morphology of the cEG cells is very similar to the cES cells (FIG. 5A,B). When cEG cells are injected into Stage X (E-G&K) embryos, they have the ability to colonize somatic tissues and make chimeras that, as juveniles, appear identical to chimeras made with cES cells (FIG. 6).

### Example 6

### Cultured PGCs Give Rise to Functional Gametes

PGCs that were cultured for 40 days or 110 days were injected into Stage 15 (H&H) White Leghorn embryos and 23 chicks have hatched. All of these chicks are phenotypically White Leghorns. The males were reared to sexual maturity and have been mated to Barred Rock hens (Table 1). The rate of germline transmission of the roosters varied from <1% to 87% (Table 1 and FIG. 7).

TABLE 1
The rate of germline transmission of roosters, injected with PGCs
that had been cultured for 40 and 100 days.

| | Days in | | # Black | % Germline |
|---|---|---|---|---|
| | culture | # Offspring | Offspring | Transmission |
| IV7-5 | 40 | 460 | 95 | 17 |
| IV7-6 | 40 | 672 | 1 | 0.01 |
| IV7-22 | 40 | 306 | 5 | 2 |
| IV9-1 | 110 | 470 | 410 | 87 |
| IV 9-2 | 110 | 584 | 19 | 3 |
| IV 9-13 | 110 | 341 | 2 | 0.6 |
| IV 9-15 | 110 | 482 | 7 | 1.5 |
| IV 9-48 | 110 | 356 | 5 | 1.4 |

### Example 7

### Sensitivity of PGCs to Neomycin and Puromycin

The sensitivity of PGCs to puromycin and neomycin was determined to establish the concentration of puromycin and neomycin required to allow the growth of cells that express antibiotic resistance under the control of the CX-promoter which is strongly expressed in all tissues (Origen Therapeutics, unpublished). These experiments demonstrated that a concentration of 400 μg/ml neomycin for 10 days is necessary to eliminate all non-transfected cells. A concentration of 0.5 μg/ml puromycin was sufficient to eliminate PGCs within 7-10 days.

### Example 8

### Genetic Modification of PGCs

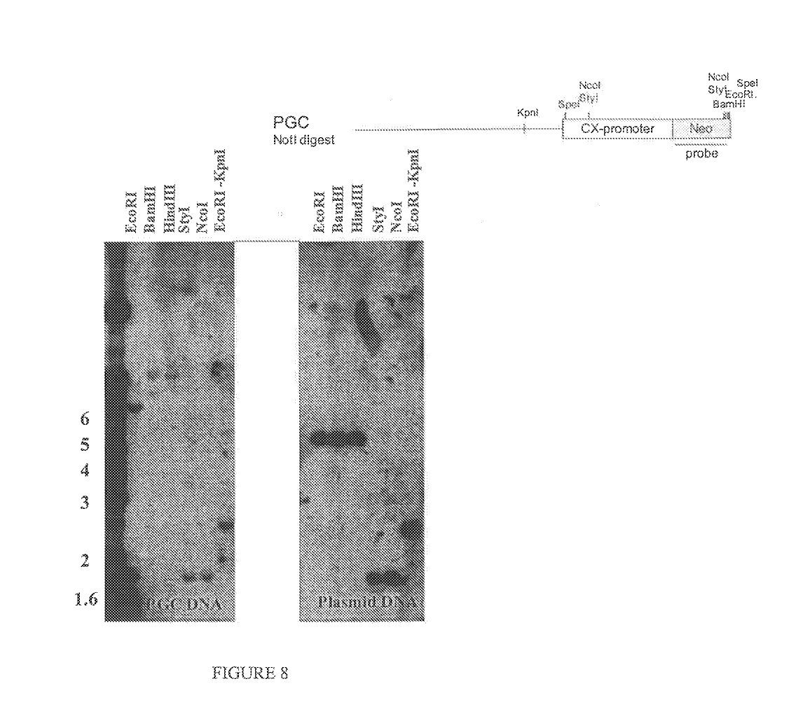

Twenty microgram (20 μl) of a NotI linearized cx-neo transgene (see FIG. 8) was added to a population of 5.8×10⁶ PGCs that had been in culture for 167 days. The cells and DNA were resuspended in 800 μl of electroporation buffer and 8 square wave pulses of 672 volts and 100 μsec duration were applied. After ten minutes, the cells were resuspended in culture medium and aliquoted into 24-well plates. Two days after electroporation, 400 μg of neomycin were added per ml of medium to select cells that were expressing the cx-neo transgene. The cells were kept under selection for 19 days. After 19 days, the cells were taken off selection and expanded for analysis. A proportion of the PGCs was kept under 400 μg/ml for another 31 days demonstrating that the PGCs were functionally resistant to the antibiotic. Referring to FIG. 8, for the plasmid control, the cx-neo plasmid DNA was linearized with NotI and then digested with EcoRI or BamHI to produce a fragment that is slightly smaller (5 kb) than the intact plasmid which is shown by the HindIII digestion. Internal fragments of approximately 2 kb of the cx-neo plasmid were released by digestion with StyI or NcoI. A larger internal fragment of approximately 2.6 kb was released by digestion with EcoRI and KpnI. Digestion of genomic DNA from the line of PGCs with EcoRI, BamHI and HindIII revealed bands that are larger than 6 kb illustrating that the cx-neo transgene was incorporated into the PGC genome. The internal fragments revealed in plasmid DNA following digestion with StyI, NcoI and EcoRI with KpnI were also present in genomic DNA from the PGCs indicating that the cx-neo transgene was integrated into the PGC genome without alteration. Using convential transgene construction techniques, additional elements can be incorporated such as regulatory elements, tissue specific promoters and exogenous DNA encoding proteins are examples. Monoclonal antibodies are preferred example of a protein encoded by exogenouse DNA and human monoclonals are preferred species thereof.

As noted above, the performance of genetic modifications in PGCs to produce transgenic animals has been demonstrated in only a very few species. Analogous genetic manipulations can be achieved in chicken PGCs by referring to those achieved using ES cells in mice. In mice, the separate use of homologous recombination followed by chromosome transfer to embryonic stem (mES) cells for the production of chimeric and transgenic offspring is well known. Powerful techniques of site-specific homologous recombination or gene targeting have been developed (see Thomas, K. R. and M. R. Capecchi, Cell 51: 503-512, 1987; review by Waldman, A. S., Crit. Rev. Oncol. Hematol. 12: 49-64, 1992). Insertion of cloned DNA (Jakobovits, A., Curr. Biol. 4: 761-763, 1994) and manipulation and selection of chromosome fragments by the Cre-loxP system techniques (see Smith, A. J. et al., Nat. Genet. 9:376-385, 1995; Ramirez-Solis, R. et al., Nature 378:720-724, 1995; U.S. Pat. Nos. 4,959,317; 6,130,364; 6,130,364; 6,091,001; 5,985,614) are available for the manipulation and transfer of genes into mES cells to produce stable genetic chimeras. Many such techniques that have proved useful in mammalian systems would be available to be applied to chicken PGCs if the necessary cultures were available.

1. A culture comprising 1×10⁵ or more chicken primordial germ cells (PGCs) whose genomes stably comprise and express an exogenous DNA sequence, wherein the PGCs are derived from culturing whole blood obtained from a stage 12-17 chicken embryo for at least 40 days in vitro, wherein the PGCs contribute to the germline of a recipient embryo, transcribe the chicken vasa homologue (CVH), and express the Cvh protein.

2. The culture of claim 1, wherein the PGCs are cultured in medium comprising buffalo rat liver (BRL) cells.

3. The culture of claim 1, where the PGCs are cultured in medium comprising fibroblast growth factor (FGF).

4. The culture of claim 1 where the PGCs are cultured in medium comprising stem cell factor.

5. The culture of claim 1 wherein the PGCs are cultured in medium comprising chicken serum.

6. (canceled)

7. (canceled)

8. A method to obtain a culture of transfected chicken primordial germ cells (PGCs) whose genomes stably comprise and express an exogenous DNA sequence, the method comprising:
obtaining whole blood comprising chicken PGCs from a stage 12-17 chicken embryo;
culturing the whole blood with passaging for at least 40 days in vitro, whereby the PGCs outgrow other cells in the culture, and the culture comprises 1×10⁵ or more PGCs;
introducing an exogenous DNA sequence into the chicken PGCs
selecting PGCs whose genomes stably comprise and express the exogenous DNA sequence by maintaining the PGCs for at least 19 days in vitro; and
expanding the PGCs whose genomes stably comprise and express the exogenous DNA sequence such that a culture of the PGCs comprises 1×10⁵ or more PGCs;
wherein the expanded culture of PGCs whose genomes stably comprise and express the exogenous DNA sequence contribute to the germline of a recipient embryo.

9. (canceled)

10. (canceled)

11. The method of claim 8, wherein the whole blood is cultured in medium comprising buffalo rat liver cells (BRL).

12. The method of claim 8, wherein the whole blood is cultured in medium comprising fibroblast growth factor (FGF).

13. The method of claim 8, wherein the whole blood is cultured in medium comprising stem cell factor.

14. The method of claim 8, wherein the whole blood is cultured in medium comprising chicken serum.

15. (canceled)

16. The method of claim 8, where the exogenous DNA sequence encodes a protein.

17. The method of claim 16 wherein the protein is a monoclonal antibody.

18. The method of claim 17, wherein the monoclonal antibody is encoded by a human polynucleotide sequence.

19. (canceled)

20. The method of claim 8, wherein the PGCs are passaged multiple times.
